(19) United States
(12) Patent Application Publication
(10) Pub. No.: US 2021/0205376 A1
(43) Pub. Date: Jul. 8, 2021

(54) NOVEL TREATMENT FOR MUCOSITIS

(71) Applicants: INSTITUT NATIONAL DE RECHERCHE POUR L'AGRICULTURE, L'ALIMENTATION ET L'ENVIRONNENT, Paris (FR); UNIVERSIDADE FEDERAL DE MINAS GERAIS, Belo Horizonte (BR); INSTITUT SUPÉRIEUR DES SCIENCES AGRONOMIQUES, AGROALIMENTAIRES, HORTICOLES ET DU PAYSAGE, Rennes (FR)

(72) Inventors: Gwénaël JAN, Rennes (FR); Yves LE LOIR, Le Rheu (FR); Florence VALENCE, Plougonvelin (FR); Vasco Ariston DE CARVALHO AZEVEDO, Belo Horizonte - MG (BR); Rodrigo DIAS DE OLIVEIRA CARVALHO, Recife - PE (BR); Fillipe Luiz ROSA DO CARMO, Belo Horizonte - MG (BR); Barbara Fernandes CORDEIRO, Belo Horizonte - MG (BR)

(21) Appl. No.: 17/273,580

(22) PCT Filed: Sep. 4, 2019
(86) PCT No.: PCT/EP2019/073626

§ 371 Date: Mar. 4, 2021

(30) Foreign Application Priority Data
Sep. 5, 2018 (FR) 1870991

(51) Int. Cl. A61K 35/741; A61P 1/02

(52) U.S. Cl. CPC A61K 35/741; A61P 1/02

(57) ABSTRACT

Disclosed is a composition including at least one bacterial strain belonging to the genus Propionibacterium for use in the treatment or prevention of mucositis in a subject, preferably for the treatment or prevention of the alteration of tight junctions of epithelial tissue associated with mucositis.

### CROSS-REFERENCE TO RELATED APPLICATIONS

This application is the U.S. national phase of International Application No. PCT/EP2019/073626 filed Sep. 4, 2019 which designated the U.S. and claims priority to FR 1870991 filed Sep. 5, 2018, the entire contents of each of which are hereby incorporated by reference.

### REFERENCE TO SEQUENCE LISTING SUBMITTED ELECTRONICALLY

The content of the electronically submitted sequence listing (Name: 7255-43_SEQ_LISTING.txt; Size: 33.5 kilobytes; and Date of Creation: Mar. 4, 2021) filed with the application is incorporated herein by reference in its entirety.

### FIELD OF THE INVENTION

The present invention relates to mucositis and, in particular, to the prevention or treatment of its two components, namely the inflammation of the epithelial tissue and the alteration of the tight junctions (zona occludens) within this epithelial tissue.

### PRIOR ART

Mucositis is a pathology defined by the presence of inflammatory and/or ulcerative lesions of the oral and/or gastrointestinal tract. While its onset may be the result of an infectious disease, immune deficiency, or drug toxicity, its occurrence usually results from cancer therapy. Thus, while 20 to 40% of patients undergoing chemotherapy are affected, it is present in more than 80% of patients undergoing high-dose chemotherapy and in almost all patients with head or neck cancer undergoing radiation therapy.

This occurrence of mucositis is closely associated with epithelial cell dysfunction and death, with involvement of endothelial cells. While the inducing mechanisms differ between chemotherapy-induced and radiation-induced mucositis, the subsequent steps show similarities. The sequence of events thus consecutively involves the appearance of reactive oxygen species, so-called clonogenic cell mortality (with a loss of proliferative capacity), the secretion of pro-inflammatory cytokines followed by apoptosis of epithelial and endothelial cells, the alteration of tight junctions (zona occludens) within epithelial tissues, and finally the formation of ulcers.

The consequences of mucositis can be dramatic since, in addition to the undernutrition it can cause in the patient, its occurrence often leads to modifying, and sometimes even interrupting, the patient's anti-cancer treatment protocol, thus impacting the patient's chances of success.

Mucositis treatment options available to the practitioner are described in LALLA et al., (MASCC/ISOO Clinical Practice Guidelines for the Management of Mucositis Secondary to Cancer Therapy, Cancer, vol. 120(10), p: 1453-1461, 2014) and are few in number.

Thus, and for the treatment of gastrointestinal mucositis, it is possible to use a) an intravenous administration of amifostine (cytoprotective agent) to try to prevent its occurrence in patients undergoing radiotherapy or b) loperamide (antidiarrheal agent) to avoid at least the occurrence of diarrhea in patients undergoing standard or high-dose chemotherapy.

Now, due to side effects or conflicting results, various other agents cannot be recommended for the treatment of mucositis, including activated charcoal, anti-inflammatory drugs such as budesonide, balsalazide, or celecoxib, cytoprotectants such as sodium folinate, antibiotics such as cefixime, levofloxacin, metronidazole, or neomycin, epithelial cell growth factors such as palifermin.

Clearly, it is difficult to treat mucositis and, more specifically, its two components: 1) inflammation of the epithelial tissue and 2) alteration of the tight junctions of the epithelial tissue. It should also be noted that it is precisely this dual component that results in the ineffectiveness of anti-inflammatory drugs in treating mucositis and allowing a return to normal epithelial tissue.

Therefore, there is a need for a new means of preventing and/or treating mucositis, especially gastrointestinal mucositis, that is able to treat both its components, namely capable of 1) reducing inflammation and 2) restoring tight junctions.

### SUMMARY OF THE INVENTION

The inventors have now demonstrated that different bacterial strains belonging to the genus Propionibacterium have, in addition to an anti-inflammatory action, an action of consolidation of tight junctions within the epithelial tissue.

Based on this observation of a potential action on the two components of mucositis, the inventors therefore tested the use of these strains on a chemo-induced mucositis. The result of these experiments was a noticeably clear improvement in the state of the subjects treated. The analysis of the associated mechanism of action also showed that the SIpB protein plays a central role in this action on the two components of mucositis.

It is true that the use of probiotics to treat certain symptoms of mucositis had been suggested along with the use of species of the genus Lactobacillus for the prevention of diarrhea alone in patients undergoing radiotherapy and chemotherapy with pelvic complications (CIORBA et al., Opin Support Palliat Care, vol. 9(2), p: 157-162, 2015). Now, this publication showed that, even for this limited and quite distinct framework of invention, there was great variability. Thus, while bacteria of the genus Lactobacillus were effective, bacteria of the genus Bifidobacterium were not. Moreover, this efficacy had shown significant inter-species, and even inter-strain, variability, with the Lactobacillus rhamnosus GG strain being particularly effective. Finally, there was no indication that, unlike the anti-inflammatory components tested so far, bacterial strains of the genus Propionibacterium could be used not for the prevention of diarrhea in patients undergoing radiotherapy and chemotherapy with pelvic complications, but to prevent or successfully treat a subject suffering from mucositis.

Accordingly, a first object of the invention relates to a composition comprising at least one bacterial strain belonging to the genus Propionibacterium for use in the treatment or prevention of mucositis in a subject, preferably for the treatment or prevention of the alteration of tight junctions of epithelial tissue associated with mucositis.

### DESCRIPTION OF THE FIGURES

FIG. 1 shows the relative expression, determined by RT-PCR, of the genes encoding different cytokines, namely interleukin 10 (A), interleukin 8 (B), interferon alpha (C), and TNF alpha (D), in HT-29 cells in contact with Lipopolysaccharides (LPS) and/or with the Propionibacterium freudenreichii 129 Wild Type (WT) strain or with the Propionibacterium freudenreichii 129 ΔslpB (ΔslpB) deleted mutant strain. These measurements were made in triplicate on three independent cultures per conditions. The averages and standard deviations are therefore calculated for 9 measurements per condition. Asterisks represent the statistically significant difference between the strains: * p<0.05; ** p<0.01; *** p<0.001; **** p<0.0001.

FIG. 2 shows the absolute number and frequency of CD4+ RORγT+ cells (A) and CD4+ FOXP3+ cells (B) in the spleen of mice suffering from mucositis induced by an injection of 5-FU (the control group was injected with saline), and having received a treatment based on water, YEL medium, the Propionibacterium freudenreichii Wild-type (WT) bacterium, or the Propionibacterium freudenreichiiΔslpB (ΔslpB) deleted mutant strain. The frequencies were measured by flow cytometry. The averages and standard deviations were calculated based on 5 mice per group. Asterisks represent the statistically significant difference between the strains: * p<0.05; ** p<0.01; *** p<0.001; **** p<0.0001.

FIG. 3 shows the assay of different cytokines by ELISA in mice suffering from mucositis induced by an injection of 5-FU (the control group was injected with saline), and having received a treatment with water, YEL medium, the Propionibacterium freudenreichii wild-type (WT) bacterium, or the Propionibacterium freudenreichii ΔslpB (ΔslpB) deleted mutant strain. The cytokines studied are Interleukin-10 (FIG. 3A), Interleukin-12 (FIG. 3B), Interleukin-1β (FIG. 3C). In addition, the IL-10/IL-12 ratio is shown in FIG. 3D. The averages and standard deviations were calculated based on triplicate measurements on 3 independent replicates of ilea of 6 animals per group. Asterisks represent the statistically significant difference between the strains: * p<0.05; ** p<0.01; *** p<0.001; **** p<0.0001.

FIG. 4 shows the relative expression level of Claudine 1 mRNA, measured by RT-PCR, in mice suffering from mucositis induced by an injection of 5-FU (the control group was injected with saline), and having received a treatment based on water, YEL medium, the Propionibacterium freudenreichii Wild-type (WT) bacterium, or the Propionibacterium freudenreichii ΔslpB (ΔslpB) deleted mutant strain. The averages and standard deviations were calculated based on triplicate measurements on 3 independent replicates of ilea of 6 animals per group. Asterisks represent the statistically significant difference between the strains: * p<0.05; ** p<0.01; *** p<0.001; **** p<0.0001.

FIG. 5 shows sections of ileum from mice suffering from mucositis induced by an injection of 5-FU (the control group was injected with saline), and treated with water, YEL medium, the Propionibacterium freudenreichii Wild-type (WT) bacterium, or the Propionibacterium freudenreichii ΔslpB (ΔslpB) deleted mutant strain (FIG. 5B). The ileum sections are stained with hematoxylin and eosin, and correspond to a 20× objective magnification. The scale bar corresponds to 100 μm. Their histological scores associated with inflammation are shown in FIG. 5A. The averages and standard deviations were calculated based on 18 ileum sections per group (3 independent ileum sections of 6 mice per group). Asterisks represent the statistically significant difference between the strains: * p<0.05; ** p<0.01; *** p<0.001; **** p<0.0001.

FIG. 6 shows the villi height (FIG. 6A), the ratio of villi height to crypt depth (FIG. 6B), and the granular density of Paneth cells (FIG. 6C) of mouse ilea with mucositis induced by an injection of 5-FU (the control group was injected with saline), and having received a treatment based on water, YEL medium, the Propionibacterium freudenreichii Wild-type (WT) bacterium, or the Propionibacterium freudenreichii ΔslpB (ΔslpB) deleted mutant strain. The microscopic morphometric analysis of Paneth cell granules was performed based on 10 images of ilea at 40× objective magnification. The averages and standard deviations were calculated based on 18 ileum sections per group (3 independent ileum sections of 6 mice per group). Asterisks represent the statistically significant difference between the strains: * p<0.05; ** p<0.01; *** p<0.001; **** p<0.0001.

FIG. 7 shows the intestinal permeability measured 72 h after induction of mucositis. The radioactivity of DTPA-TC99m was measured in the blood of mice. The averages and standard deviations were calculated based on an independent measurement for 5 mice per group. Asterisks represent the statistically significant difference between the strains: * p<0.05; ** p<0.01; *** p<0.001; **** p<0.0001.

FIG. 8 shows the weight loss observed in mice after induction of mucositis by injection of 5-FU on different groups of mice. The averages and standard deviations were calculated based on 18 ileum sections per group (3 independent ileum sections of 6 mice per group). Asterisks represent the statistically significant difference between strains: * p<0.05; ** p<0.01; *** p<0.001; **** p<0.0001.

### DETAILED DESCRIPTION OF THE INVENTION

In the context of the use according to the invention, said at least one bacterial strain belonging to the genus Propionibacterium is envisaged as a probiotic.

By “probiotic”, within the meaning of the present invention, is meant living or dead microorganisms which, when administered in sufficient quantity to a host, in particular an animal, have a beneficial effect on the health of the host.

In the context of the use according to the invention, the bacterial strain belonging to the genus Propionibacterium can be administered to humans or animals without risk to their health, which constitutes an undeniable advantage.

By bacterial strain belonging to the genus Propionibacterium, is meant a bacterial strain selected from the group consisting of P. freudenreichii, P. thoenii, P. jensenii, and P. acidipropionici, preferably the bacterial strain belonging to the genus Propionibacterium belongs to the species P. freudenreichii.

This bacterial strain advantageously expresses the SIpB (surface layer protein B) protein, preferably it expresses an SIpB protein from the species P. freudenreichii.

By way of example of the SIpB protein of the species P. freudenreichii, proteins with the accession numbers WP_051733954.1 (SEQ id No. 1), WP_055345346.1 (SEQ id No. 2), CEG94741.1 (SEQ id No. 3), SCQ66536.1 (SEQ id No. 4), SBT28366.1, WP_097850726.1 (SEQ id No. 5), WP_060760754.1 (SEQ id No. 6), and WP_060763255.1 (SEQ id No. 7), can be mentioned.

By “mucositis”, is meant a pathology defined by the presence of inflammatory and/or ulcerative lesions of the oral and/or gastrointestinal tract. Preferably, the mucositis is a gastrointestinal mucositis.

Preferably still, the use according to the invention is for the treatment or prevention of the alteration of tight junctions of epithelial tissue associated with mucositis.

By “subject”, within the meaning of the present invention, is meant an animal, preferably a mammal, and even more preferably a human. It should be noted that it corresponds more particularly to a subject undergoing or about to undergo treatment by radiotherapy or chemotherapy.

Preferably, the strain of the invention may be used in association with one or more other probiotic species for the preparation of probiotic compositions.

They can be used in the form of whole bacteria, alive or not, and also in the form of bacterial lysate, or in the form of bacterial fractions.

According to a preferred embodiment, the composition of the invention comprises at least 10⁶, preferably at least 10⁷, and at most 5×10¹⁰ bacteria belonging to the genus Propionibacterium per mL.

Advantageously, the composition volume is at least one mL. Now, the administration of the composition according to the invention can go up to 500 mL, or even one liter.

Preferably, the composition of the invention comprises one or more acceptable carriers.

By “carrier”, within the meaning of the present invention, is meant any substance, other than the active ingredient of the probiotic composition, intended to impart to said probiotic composition particular physical, chemical, or taste characteristics not interacting with the active ingredient. Said carriers may be of natural or synthetic origin.

The one skilled in the art will be able, based on his/her knowledge, to identify an acceptable carrier for a probiotic composition. Examples of acceptable carriers include: sugars such as sucrose, glucose, fructose, palatinose, trehalose, lactose, and xylose; polyols such as sorbitol, xylitol, erythritol, and lactitol; emulsifiers such as sucrose esters of fatty acids, glycerol esters of fatty acids, and lecithin; thickeners and stabilizers such as carrageenan, xanthan gum, guar gum, pectin, and locust bean gum; acidifiers such as citric acid, lactic acid, and malic acid; fruit juices such as lemon juice, orange juice, and berry juice; vitamins such as vitamin A, vitamin B, vitamin C, vitamin D, and vitamin E; and minerals such as calcium, iron, manganese, and zinc.

According to another preferred embodiment, the composition of the invention is in a form that can be administered orally.

Such compositions may take the form of capsules, tablets, powders, lozenges, granules, soft capsules, reconstitutable powders, suspensions, gels, frozen compositions, oral liquid preparations, or conventional food products.

Preferably, the composition of the invention is in the form of a capsule.

Oral tablets and capsules may be a single dose and may contain one or more acceptable carriers such as binders, fillers, lubricants, wetting agents, or disintegrating agents. The tablets can be coated by methods well known in the pharmaceutical field.

Liquid oral preparations may, for example, be in the form of aqueous or oily suspensions, solutions, emulsions, syrups, or elixirs in the form of dehydrated products reconstitutable with water or any other acceptable vehicle. Such liquid preparations may contain commonly used additives such as suspending agents, emulsifiers, non-aqueous vehicles, preservatives, and possibly flavorings and/or colorings.

Alternatively, the composition of the invention is in the form of a food product or a food supplement.

By “food product” and “conventional food product”, within the meaning of the present invention, is meant any substance or product, whether processed, partially processed, or unprocessed, intended to be ingested by humans or animals. Food products can be of animal, vegetable, or mineral origin. Food products provide the body with the energy and nutrients it needs to function. The food product according to the invention may be a liquid, hence a drink, or a solid.

By “food supplement”, within the meaning of the present invention, is meant any product containing nutrients that are beneficial to health and that are added to the normal diet of humans or animals. Food supplements contain, but are not limited to, vitamins, minerals, amino acids, enzymes, and probiotics.

The composition of the invention may be in the form of a dairy product, for example based on cow's, goat's, or sheep's milk, which may be in liquid, solid, or powder form (milk, fermented milk, butter, cheese, cream, etc.).

By “prevent” and “prevention”, within the meaning of the present invention, is meant avoiding the occurrence of a disease, disorder, or one or more signs and/or symptoms of a disease or disorder.

By “treat” and “treatment”, within the meaning of the present invention, is meant improving or remedying a disease, disorder, or one or more signs and/or symptoms of a disease or disorder.

The invention also comprises a method of preventing and/or treating mucositis, comprising a step of administering to a subject a therapeutically effective amount of at least one bacterial strain of Propionibacterium freudenreichii.

By “therapeutically effective amount”, within the meaning of the present invention, is meant the minimum amount necessary to obtain the effect expected from the administration of such a strain or composition.

According to a preferred embodiment, the composition of the invention comprises at least 10⁶, preferably at least 10⁷, and at most 5×10¹⁰ bacteria belonging to the genus Propionibacterium per mL.

Now, the different preferred embodiments of the method according to the invention are the same as for the use according to the invention.

The following examples are provided by way of illustration and shall not limit the scope of this invention.

### Examples

1. Study of the Anti-Inflammatory Effect of Propionibacterium freudenreichii

a. In Vitro Study of the Anti-Inflammatory and Tight Pro-Junction Effect of Propionibacterium freudenreichii on HT-29 Intestinal Cells with LPS-Induced Inflammation

The P. freudenreichii CIRM-BIA 129 (WT) wild strain and the P. freudenreichii CIRM-BIA 129ΔslpB (CB129ΔslpB) mutant strain (do Carmo et al., 2017) were cultured at 30° C. in a YEL culture medium comprising a yeast extract and lactate. For the CB129ΔslpB mutant strain, the YEL culture medium is supplemented with chloramphenicol (10 μg·mL−1). The growth of P. freudenreichii strains is then monitored with a spectrophotometer by following the optical density of the cultures at 650 nm (OD650 nm) and also by determining the number of colony forming units (CFUs) by culturing a given volume of inoculated YEL medium.

In order to determine their potential activity on intestinal tissue, these two bacterial strains were brought into contact with human intestinal epithelial cells of the HT-29 lineage.

To do this, HT-29 cells were cultured in T-25 flask until confluence (10⁶ cells/mL). The culture medium was then renewed with an antibiotic-free culture medium and the cells (one million cells per well) were either co-incubated for 7 hours or not with a medium either comprising lipopolysaccharide (LPS) at 100 ng/mL or not and in the presence or not of bacteria (10 million bacteria per well) of the wild-type WT strain or of the CB129ΔslpB mutant strain (YEL medium alone was used as a control).

After a 7-hour co-culture, the cellular RNAs were then isolated using the TRIZOL (INVITROGEN AMBION) reagent following the manufacturer's recommendations. Based on these cellular RNAs, complementary DNAs were then synthesized using the QSCRIPT CDNA SYNTHESIS kit (QUANTA BIOSCIENCES), again following the manufacturer's instructions. The determination of the expression of different cellular genes was then performed by real-time PCR, using gene-specific probes, on a CFX96 system (BIO-RAD), and the quantification of the mRNA level of the targeted genes was performed using the CFX Manager™ software. For information, the RNA expression levels were normalized to the expression levels of GAPDH and actin.

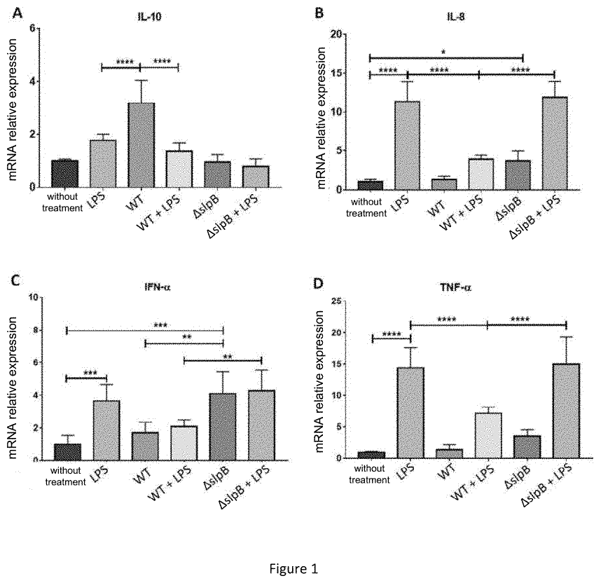

The results showed a strong induction of interleukin 10 expression in HT-29 cells in response to co-incubation with P. freudenreichii CIRM-BIA 129 (WT) and in the absence of LPS, which cytokine has a strong immunomodulatory activity (FIG. 1A). It should be noted that this induction is absent in the presence of the CB129ΔslpB mutant strain. The SIpB protein is therefore critical for this induction.

Similarly, the results show a strong induction of pro-inflammatory IL-8 cytokines (FIG. 1B; relative expression of mRNA: 11.36±2.56; significantly p<0.0001), IFN-α (FIG. 1C), and TNF-α (FIG. 1D) in the presence of LPS or LPS with the CB129ΔslpB mutant strain. In parallel, the expression level of these cytokines in HT-29 cells in the presence of LPS is almost normal in the presence of the P. freudenreichii WT wild strain.

It should be noted that the results also unexpectedly showed that the expression of claudin-1, which is a critical protein for the structuring of tight junctions, is increased for cells cultured in the presence of the wild strain.

b. In Vivo Study of the Impact of Propionibacterium freudenreichii on T Lymphocyte Subpopulations in Mice

To test this hypothesis, the inventors used female BALB/c mice between 6 and 8 weeks of age, which were kept in a temperature-controlled atmosphere with normal access to water and food.

For the treatment, the mice were given continuous access for 10 days to a diet comprising either YEL culture medium, or a culture of propionibacteria on the same YEL culture medium, containing either 10⁹ CFU/mL of bacteria of the P. freudenreichii wild strain or the P. freudenreichii CB129ΔslpB mutant strain. In order to induce mucositis, the mice then received a single intraperitoneal injection of 5-FU (300 mg/kg) on day 11, and were finally euthanized on day 14. As a control, a group of mice received an injection of saline solution (0.9% NaCl).

The distribution of T lymphocyte subpopulations in the spleen of mice is measured by flow cytometry. 10⁶ cells are isolated from mouse spleen and resuspended in a PBS solution comprising 0.2% BSA and 0.1% NaN₃ pH 7.4. To label cell surface antigens, cells are incubated with CD4 monoclonal antibodies (GK 1,5 APC EBIOSCIENCE) for 30 minutes at 4° C. The cells are then fixed and permeabilized using the FIXATION/PERMEABILIZATION WORKING SOLUTION (EBIOSCIENCE) for 1 hour prior to incubation with ALEXA FLUOR 488 RAT ANTI-MOUSE FOXP3 antibodies (BD PHARMINGEN) or PE ANTI-MOUSE RORγT antibodies (BD PHARMINGEN) for 30 minutes at 4° C. After washing, the cells are sorted using a FACS CALIBUR cytometer (BECTON DICKINSON). The T cells are first isolated based on the CD4-positive label, and then RORγ-T positive and FOXP3-positive cells are selected and counted.

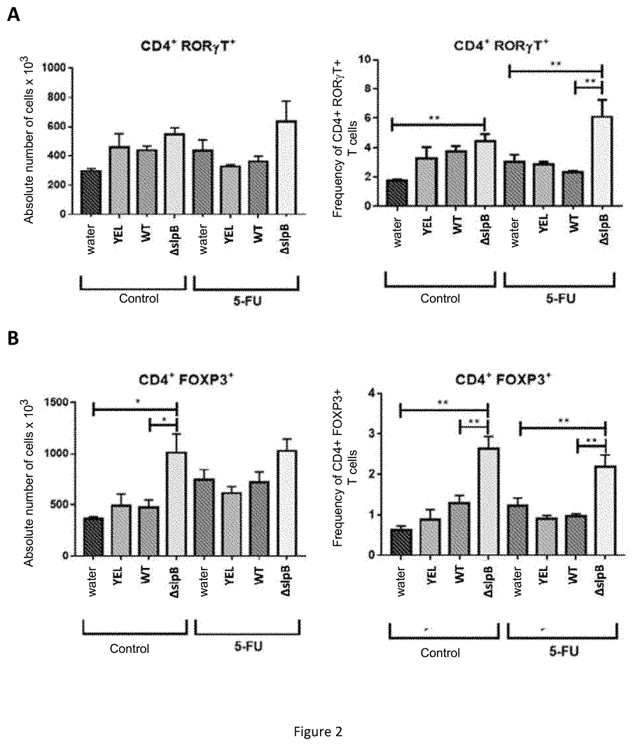

As shown in FIG. 2, consumption of P. freudenreichii ΔslpB results in a significant increase in the frequency of RORγT-positive and FOXP3-positive T cells. This increase is not observed when Propionibacterium freudenreichii WT is consumed.

RORγT-positive and FOXP3-positive T cells are implicated in chronic inflammatory bowel diseases such as ulcerative colitis or Crohn's disease. It is possible that after consumption of P. freudenreichii ΔslpB, naive T cells may begin to express RORγT and induce a pro-inflammatory response via the Th17/IL-17A pathway. Interleukin 17A can modulate the activation and recruitment of neutrophils in the ileum. In addition, the P. freudenreichii ΔslpB mutant strain induces an increase in the CD4+ Foxp3+ regulatory T cell subpopulation. CD4+ regulatory T cells expressing Foxp3 are very abundant in the intestinal mucosa, and their proliferation appears to be part of a mechanism to control inflammation mediated by Th17 effector cells.

Thus, Propionibacterium freudenreichii ΔslpB induces the production of pro-inflammatory Th17 cells in the spleen of mice, unlike the Propionibacterium freudenreichii WT strain.

c. In Vivo Study of the Impact of Propionibacterium freudenreichii on Cytokine Production in the Ileum of Mice

To determine the expression of cytokines by ELISA, ilea were collected, weighed, and homogenized in a PBS solution comprising 0.05% TWEEN-20, 0.1 mM benzethonium chloride, 0.1 mM phenylmethylsulfonyl fluoride, 10 mM EDTA, and 20 U aprotinin. After homogenization of the material, the samples were centrifuged at 3000 g for 10 minutes and the supernatant was then collected for cytokine measurements by the ELISA method. These supernatants were added to microtiter plates previously coated with antibodies to IL-10, IL-12, or Il-1β. After overnight incubation, cytokine binding was revealed with biotinylated monoclonal antibodies to these specific cytokines according to the manufacturer's instructions.

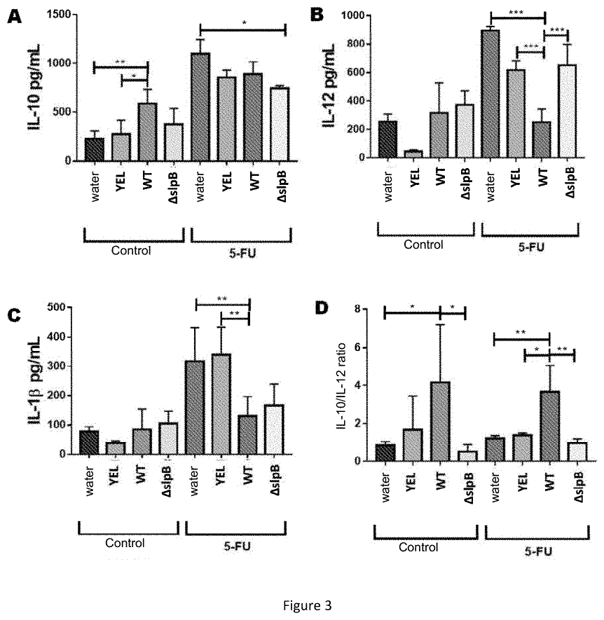

The results of the quantification of cytokines by ELISA are shown in FIG. 3.

The results showed that, apart from the 5-FU treatment, consumption of the P. freudenreichii wild strain increased IL-10 production as observed in culture on HT-29 cells, which is not possible with the P. freudenreichii CB129ΔslpB mutant strain (FIG. 3A). Following 5-FU treatment of animals, which normally induces mucositis, an increase in IL-12 and IL-1β expression is observed, which is indicative of the inflammatory state of the ileum. Consumption of the P. freudenreichii wild strain prevents the induction of expression of the pro-inflammatory cytokines IL-12 and IL-1β in mice with mucositis, compared to the control (YEL medium), as shown in FIGS. 3B and 3C.

Finally, FIG. 3D shows that a treatment comprising the P. freudenreichii WT wild-type strain significantly increases the IL-10/IL-12 ratio in mice with mucositis, in contrast to a treatment comprising the P. freudenreichii ΔslpB mutant strain, which is characteristic of a decrease in inflammation.

Consequently, the results show an immunosuppressive action of the P. freudenreichii wild strain under normal culture conditions and an anti-inflammatory action of the latter under inflammatory conditions. It should be noted that these actions are obviously mediated by the SIpB protein, since they are absent from the CB129ΔslpB mutant strain. Finally, the inventors found that, unexpectedly, the P. freudenreichii wild strain induced the expression of claudine 1 and would likely contribute to the tightening of tight junctions in the epithelial tissue. Therefore, and in view of the two components of mucositis, which are 1) inflammation of the epithelial tissue and 2) alteration of the tight junctions within the epithelial tissue, this discovery places the P. freudenreichii wild strain as an ideal candidate for the treatment and/or prevention of mucositis.

2. Study of the Tight Pro-Junction Effect of Propionibacterium freudenreichii on the Ileum Mucosa in Mice Treated with 5-FU

a. In Vivo Study of the Impact of Propionibacterium freudenreichii on the Expression of Tight Pro-Junction Genes in the Ileum in Mice

To determine the expression of different genes in the ileum, small fragments (1 cm) of these genes were also collected and stored in RNALATER (AMBION) at −80° C. prior to RNA extraction. The total RNA was then extracted with a RNEASY kit (QIAGEN) and the residual genomic DNA was digested and removed using DNase I. The corresponding complementary DNAs were then synthesized using the HIGH CAPACITY CDNA REVERSE TRANSCRIPTION KIT (APPLIED BIOSYSTEMS) according to the manufacturer's recommendations. Finally, a quantitative PCR was performed using the ITAQ UNIVERSAL SYBR GREEN mixture (BIORAD) and specific primers of the genes to be analyzed on an ABI PRISM 7900 HT system (APPLIED BIOSYSTEM) according to the manufacturer's recommendations once again.

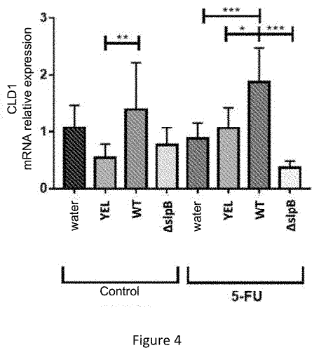

As for HT-29 cells, the results confirmed, once again, the significant increase in the level of claudin-1 expression (see FIG. 4) only in mice injected with 5-FU and having consumed the P. freudenreichii wild strain, which suggests a strengthening of the tight junctions of the epithelial tissue in these mice.

b. In Vivo Study of the Impact of Propionibacterium freudenreichii on the Histology of the Ileum Mucosa

To analyze this time the morphology of the ileum in the different groups of mice, the distal portion of the ileum was taken from each group and washed with a PBS solution before fixation in a 4% paraformaldehyde solution. The material is then embedded in paraffin. Finally, 4 μm slices are made for histological analysis. The samples are stained with haematoxylin and eosin. A histological inflammation score is determined based on the measurement of the three major histological changes induced by mucositis: (i) intensity of infiltration of mononuclear and polynuclear cells into the lamina propria, (ii) presence of ulceration and erosion, and (iii) alteration of mucosal structure. A score is assigned according to the severity of tissue damage: (0) no lesions; (1) mild; (2) moderate; (3) severe. A morphometric analysis is also performed based on the analysis of ten images of the ileum of each mouse. The granular density of Paneth cells is determined by evaluating the intracellular volume occupied by the secretory granules. Villi size and crypt depth are measured from the tip of the villus to the base of the adjacent crypt, allowing the calculation of a villi size/crypt depth ratio.

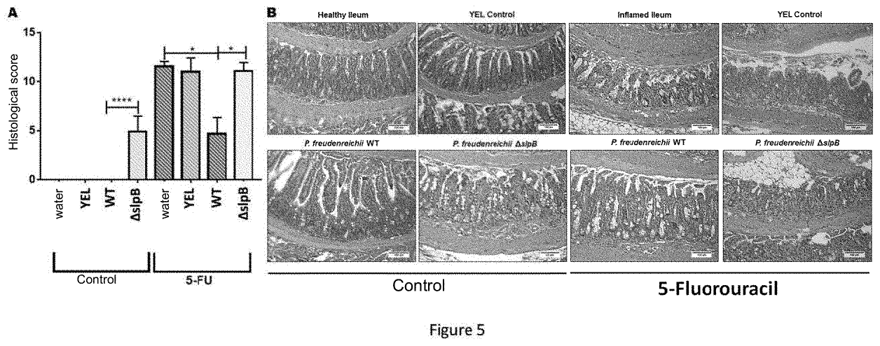

The results (shown in FIG. 5) show, in the control group, clear changes in the morphological structure of the ileum with an increase/thickening of the submucosal layer and the muscle layer, an increase in the number of inflammatory cells and a decrease/shortening of villi, a thinning of the epithelium and an increase in the number of mononuclear or polynuclear inflammatory cells infiltrated into the lamina propria (FIG. 5). Consumption of the P. freudenreichii wild strain reduces both infiltration of inflammatory cells and alterations of the intestinal mucosa (in particular by partially preventing the reduction of villi), which induces a lower histological score than negative controls, which the P. freudenreichii CB129ΔslpB mutant strain does not do.

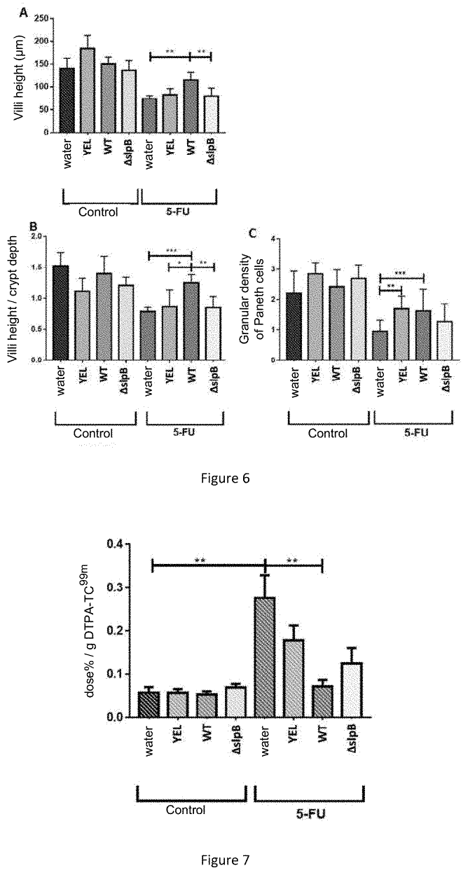

On the other hand, a reduction in villi height was observed in mice treated with 5-FU, which was significantly limited by the consumption of P. freudenreichii WT (114 μm±17.92; p>0.0001) (FIG. 6A). Such an effect on the reduction of villi height is not observed with consumption of P. freudenreichii ΔslpB.

Thus, consumption of P. freudenreichii WT preserves villi architecture by increasing villi height and the villi size/crypt depth ratio in mice treated with 5-FU (FIGS. 6A and 6B).

Regarding the granular density of Paneth cells, this is reduced during a mucositis induced by injection of 5-FU. Now, the consumption of P. freudenreichii WT allows this reduction to be limited (FIG. 6C).

The results therefore show that the consumption of Propionibacterium feudenreichii WT improves the preservation of the mucosa of the ileum of mice with mucositis caused by 5-FU.

c. In Vivo Study of the Impact of Propionibacterium freudenreichii on Intestinal Permeability

Intestinal permeability is measured 72 hours after 5-FU-induced mucositis. Mice were administered by gavage 0.1 mL of diethylene triamine pentaacetic acid (DTPA) labeled with 18.5 MBq of Technetium 99m. 4 hours after gavage, blood is collected and radioactivity is measured, for calculating a percentage dose per gram of blood according to the equation: % dose/g blood=(cpm in g blood/standard cpm)×100 cpm (cpm=radioactivity counts per minute).

The results show that the injection of 5-FU induces a strong increase in intestinal permeability (FIG. 7). Now, the consumption of P. freudenreichii WT limits this increase significantly (p>0.01). It should be noted that this is not observed when consuming P. freudenreichii ΔslpB.

Thus, consumption of Propionibacterium freudenreichii WT prevents intestinal permeability in mice with 5-FU-induced mucositis.

d. In Vivo Study of the Impact of Propionibacterium freudenreichii on the Weight of Mice Treated with 5-FU

The effect of probiotic intake on mucositis-induced weight loss following 5-FU administration in mice is studied. The weight of the mice is measured before and after the injection of 5-FU.

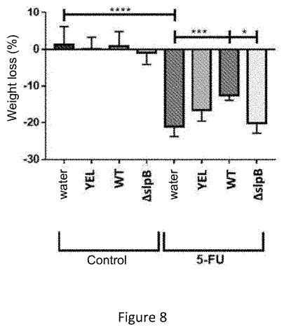

The results are shown in FIG. 8. A significant weight reduction was observed in mice injected with 5-FU (Figure xxA). Now, the results show that consumption of P. freudenreichii WT significantly limited this weight loss (loss of 13%±1.15) compared to the negative controls (loss of 20.94%±3.21). Now, the consumption of P. freudendenreichii ΔslpB does not allow such a limitation of weight loss (19.34%±2.58 compared to the P. freudenreichii WT group, p<0.05).

Consequently, the results establish that the P. freudenreichii wild strain is capable of acting on both components of mucositis, which action is obviously mediated by the SIpB protein.

Finally, this activity is confirmed by monitoring the weight of mice, which shows that consumption of the P. freudenreichii wild strain allows weight loss in animals injected with 5-FU to be significantly reduced, whereas the mutant does not have this protective effect.

In conclusion, the results demonstrate the possible use of a bacterial strain belonging to the genus Propionibacterium for the treatment or prevention of mucositis in a human subject.

1. A method of preventing and/or treating mucositis, comprising a step of administering to a subject a therapeutically effective amount of at least one bacterial strain belonging to the genus Propionibacterium.

2. The method of claim 1, wherein said method is for the treatment or prevention of the alteration of tight junctions of epithelial tissue associated with mucositis.

3. The method of claim 1, wherein the bacterial strain belonging to the genus Propionibacterium is selected from the group comprising P. freudenreichii, P. thoenii, P. jensenii, and P. acidipropionici.

4. The method of claim 1, wherein the bacterial strain belonging to the genus Propionibacterium expresses the SlpB (surface layer protein B) protein.

5. The method of claim 1, wherein the bacterial strain belonging to the genus Propionibacterium belongs to the species Propionibacterium freudenreichii.

6. The method of claim 1, wherein the mucositis is a gastrointestinal mucositis.

7. The method of claim 1, wherein the subject is a mammal.

8. The method of claim 1, wherein said subject is undergoing or is about to undergo treatment by radiotherapy or chemotherapy.

9. The method of claim 1, wherein said at least one bacterial strain belonging to the genus Propionibacterium is either alive or not.

10. The method of claim 1, wherein said composition comprises at least 10⁶ bacteria belonging to the genus Propionibacterium per mL.

11. The method of claim 3, wherein the bacterial strain belonging to the genus Propionibacterium expresses the SlpB protein from the species P. freudenreichii.

12. The method of claim 7, wherein the subject is a human.

13. The method of claim 10, wherein said composition at least 10⁷ bacteria belonging to the genus Propionibacterium per mL.

14. The method of claim 10, wherein, said composition comprises at most 5×10¹⁰ bacteria belonging to the genus Propionibacterium per mL.

15. The method of claim 2, wherein the bacterial strain belonging to the genus Propionibacterium is selected from the group comprising P. freudenreichii, P. thoenii, P. jensenii, and P. acidipropionici.

16. The method of claim 2, wherein the bacterial strain belonging to the genus Propionibacterium expresses the SlpB (surface layer protein B) protein.

17. The method of claim 3, wherein the bacterial strain belonging to the genus Propionibacterium expresses the SlpB (surface layer protein B) protein.

18. The method of claim 2, wherein the bacterial strain belonging to the genus Propionibacterium belongs to the species Propionibacterium freudenreichii.

19. The method of claim 3, wherein the bacterial strain belonging to the genus Propionibacterium belongs to the species Propionibacterium freudenreichii.

20. The method of claim 4, wherein the bacterial strain belonging to the genus Propionibacterium belongs to the species Propionibacterium freudenreichii.


SEQUENCE LISTING (7 sequences)

SEQ ID NO: 1 - protein, 556 aa, Propionibacterium freudenreichii
MSVRKSLTGMALGLALTITPLAGAVPASADTAPAPKDAITKAADWLVNDYNTNCLGDKQTSYSCSNGGLADVILALSSTGDAKYADEISTMMTNLAPQVASYTKDNAGATAKIIITVIAAHQKPSAFGGNDLVGQLQALNAENPAGGGAWGPQLSMVALTRAGETVPEALIDATVDKQNSKGGFGWGGDTGDGDNTAIGMMATAAVAKGNPRAADSLAKAVAWAQDPANLTTDDTGSYWTNYSPTNTAGMMLMAIGDVNDPKIDVSKQMDFLIGRQLPSGAFSNTLKGTNDNAMATIQALQGLTMHGYLTASAGQKNDPGTGGGTTDPGTGGGTGGGSTGGGSTGGGGSTGGGGSTGGGGSTGGGGVVTPPVTQAFTDVAPSNMYFTEIQWAAANNVTTGWKNADGTASFRPLDTTHRDAMAAFLYRLSGSPSYTAPATSPFTDVNPSNQFYKEICWLASQNITTGWPDGSFRPLDNVNRDAMAAFLYRYSQVSGFQAPAASPFADVTPGSQFYTEMSWLSANGISTGWPDQTFRPVTPIARDAMITFIYRMKHAS

SEQ ID NO: 2 - protein, 556 aa, Propionibacterium freudenreichii
MSVRKSLTGMALGLALTITPLAGAVPASADTAPAPKDAITKAADWLVNDYNTNCLGDKQTSYSCSNGGLADVILALSSTGDAKYADEISTMMANLAPQVAGYTKDNAGATAKIIITAIAAHQKPSAFGGNDLVGQLQALNAENPAGGGAWGPQLSVVALTRAGETVPEAVVDATIAKQNSKGGFGWGGDTGDGDNTAIGMMATAAVAKGNPKAADSLAKAVGWAQDPANLTTDDTGSYWTNYSPTNTAGMMLMAIGDVNDPKIDVSKQMDFLIGRQLPSGAFSNTLKGTNDNAMATTQALQGLTMHGYLTASAGQKTDPGTGGGTTDPGTGGGTGGGSTGGGSTGGGGSTGGGGSTGGGGSTGGGGVVTPPVTQAFTDVAPSNMYFTEIQWAAANNVTTGWKNADGTASFRPLDTTHRDAMAAFLYRLSGSPSYTAPATSPFTDVNPSNQFYKEICWLASQNITTGWPDGSFRPLDNVNRDAMAAFLYRYSQVSGFQAPAASPFADVTPGSQFYTEMSWLSANGISTGWPDQTFRPVTPIARDAMITFIYRMKHAS

SEQ ID NO: 3 - protein, 556 aa, Propionibacterium freudenreichii
MSVRKSLTGMALGLALTITPLAGAVPAAADTAPAPKDAITKAADWLVNDYNTNCLGDKQTSYSCSNGGLAEVILALSSTGDAKYADEISTMMANLAPQVAGYTKDNAGATAKIIITAIAAHQKPSAFGGNDLVGQLQALNAENPAGGGAWGPQLSVVALTRAGETVPEAVVDATIAKQNSKGGFGWGGDTGDGDNTAIGMMATAAVAKGNPKAADSLAKAVGWAQDPANLTTDDTGSYWTNYSPTNTAGMMLMAIGDVNDPKIDVSKQMDFLIGRQLPSGAFSNTLKGTNDNAMATTQALQGLTMHGYLTASAGQKTDPGTGGGTTDPGTGGGTGGGSTGGGSTGGGGSTGGGGSTGGGGSTGGGGVVTPPVTQAFTDVAPSNMYFTEIQWAAANNVTTGWKNADGTASFRPLDTTHRDAMAAFLYRLSGSPSYTAPATSPFTDVNPSNQFYKEICWLASQNITTGWPDGSFRPLDNVNRDAMAAFLYRYSQVSGFQAPAASPFADVTPGSQFYTEMSWLSANGISTGWPDQTFRPVTPIARDAMITFIYRMKHAS

SEQ ID NO: 4 - protein, 556 aa, Propionibacterium freudenreichii
MSVRKSLTGMALGLALTITPLAGAVPASADTAPAPKDAITKAADRLVNDYNTNCLGDKQTSYSCSNGGLADVILALSSTGDAKYADEISTMMANLAPQVAGYTKDNAGATAKIIITAIAAHQKPSAFGGNDLVGQLEALNAENPAGGGAWGPQLSVVALTRAGETVPEAVVDATIAKQNSKGGFGWGGDTGDGDNTAIGMMATAAVAKGNPKAADSLAKAVGWAQDPANLTTDDTGSYWTNYSPTNTAGMMLMAIGDVNDPKIDVSKQMDFLIGRQLPSGAFSNTLKGTNDNAMATTQALQGLTMHGYLTASAGQKTDPGTGGGTTDPGTGGGTGGGSTGGGSTGGGGSTGGGGSTGGGGSTGGGGVVTPPVTQAFTDVAPSNMYFTEIQWAAANNVTTGWKNADGTASFRPLDTTHRDAMAAFLYRLSGSPSYTAPATSPFTDVNPSNQFYKEICWLASQNITTGWPDGSFRPLDNVNRDAMAAFLYRYSQVSGFQAPAASPFADVTPGSQFYTEMSWLSANGISTGWPDQTFRPVTPIARDAMITFIYRMKHAS

SEQ ID NO: 5 - protein, 580 aa, Propionibacterium freudenreichii
MDASSAPMPPSAPGSPCGPSSWTTHERFPFMSVRKSLTGMALGLALTITPLAGAVPASADTAPAPKDAITKAADWLVNDYNTNCLGDKQTSYSCSNGGLADVILALSSTGDAKYADEISTMMANLAPQVAGYTKDNAGATAKIIITAIAAHQKPSAFGGNDLVGQLQALNAENPAGGGAWGPQLSVVALTRAGETVPEAVVDATIAKQNSKGGFGWGGDTGDGDNTAIGMMATAAVAKGNPKAADSLAKAVGWAQDPANLTTDDTGSYWTNYSPTNTAGMMLMAIGDVNDPKIDVSKQMDFLIGRQLPSGAFSNTLKGTNDNAMATTQALQGLTMHGYLTASAGQKTDPGTGGGTTDPGTGGGTGGGSTGGGSTGGGGSTGGGGSTGGGGVVTPPVTQAFTDVAPSNMYFTEIQWAAANNVTTGWKNADGTASFRPLDTTHRDAMAAFLYRLSGSPSYTAPATSPFTDVNPSNQFYKEICWLASQNITTGWPDGSFRPLDNVNRDAMAAFLYRYSQVSGFQAPAASPFADVTPGSQFYTEMSWLSANGISTGWPDQTFRPVTPIARDAMITFIYRMKHAS

SEQ ID NO: 6 - protein, 547 aa, Propionibacterium freudenreichii
MALGLALTITPLAGAVPASADTAPAPKDAITKAADWLVNDYNTNCLGDKQTSYSCSNGGLADVILALSSTGDAKYADEISTMMANLAPQVAGYTKDNAGATAKIIITAIAAHQKPSAFGGNDLVGQLQALNAENPAGGGAWGPQLSVVALTRAGETVPEAVVDATIAKQNSKGGFGWGGDTGDGDNTAIGMMATAAVAKGNPKAADSLAKAVGWAQDPANLTTDDTGSYWTNYSPTNTAGMMLMAIGDVNDPKIDVSKQMDFLIGRQLPSGAFSNTLKGTNDNAMATTQALQGLTMHGYLTASAGQKTDPGTGGGTTDPGTGGGTGGGSTGGGSTGGGGSTGGGGSTGGGGSTGGGGVVTPPVTQAFTDVAPSNMYFTEIQWAAANNVTTGWKNADGTASFRPLDTTHRDAMAAFLYRLSGSPSYTAPATSPFTDVNPSNQFYKEICWLASQNITTGWPDGSFRPLDNVNRDAMAAFLYRYSQVSGFQAPAASPFADVTPGSQFYTEMSWLSANGISTGWPDQTFRPVTPIARDAMITFIYRMKHAS

SEQ ID NO: 7 - protein, 547 aa, Propionibacterium freudenreichii
MALGLALTITPLAGAVPAAADTAPAPKDAITKAADWLVNDYNTNCLGDKQTSYSCSNGGLAEVILALSSTGDAKYADEISTMMANLAPQVAGYTKDNAGATAKIIITAIAAHQKPSAFGGNDLVGQLQALNAENPAGGGAWGPQLSVVALTRAGETVPEAVVDATIAKQNSKGGFGWGGDTGDGDNTAIGMMATAAVAKGNPKAADSLAKAVGWAQDPANLTTDDTGSYWTNYSPTNTAGMMLMAIGDVNDPKIDVSKQMDFLIGRQLPSGAFSNTLKGTNDNAMATTQALQGLTMHGYLTASAGQKTDPGTGGGTTDPGTGGGTGGGSTGGGSTGGGGSTGGGGSTGGGGSTGGGGVVTPPVTQAFTDVAPSNMYFTEIQWAAANNVTTGWKNADGTASFRPLDTTHRDAMAAFLYRLSGSPSYTAPATSPFTDVNPSNQFYKEICWLASQNITTGWPDGSFRPLDNVNRDAMAAFLYRYSQVSGFQAPAASPFADVTPGSQFYTEMSWLSANGISTGWPDQTFRPVTPIARDAMITFIYRMKHAS